(19) United States
(12) Patent Application Publication
(10) Pub. No.: US 2021/0315454 A1
(43) Pub. Date: Oct. 14, 2021

(54) MORPHOLOGY DETERMINING METHOD AND MORPHOLOGY DETERMINING SYSTEM OF CORNEAL TOPOGRAPHY

(71) Applicant: National Taiwan University, Taipei (TW)

(72) Inventors: Po-Jen SHIH, Taipei (TW); Jia-Yush YEN, Taipei (TW); I-Jong WANG, Taipei (TW); Chun-Ju HUANG, Taipei (TW); Hsin-Ju LAI, Taipei (TW)

(21) Appl. No.: 17/228,395

(22) Filed: Apr. 12, 2021

(30) Foreign Application Priority Data
Apr. 13, 2020 (TW) 109112381

(51) Int. Cl. A61B 3/10; A61B 3/16; A61B 3/00; A61B 5/00; A61B 3/14

(52) U.S. Cl. CPC A61B 3/1015; A61B 3/16; A61B 3/0025; A61B 3/165; A61B 3/14; A61B 5/7225; A61B 5/7278

(57) ABSTRACT

The present invention provides a morphology determining method of corneal topography, including: a parameter obtaining step, based on an original contour line of a cornea of a target subject as the reference, obtaining a relative displacement of each observation point of a contour line of the pressed cornea changed with time from the beginning of pressing the cornea by an external pressure to a predetermined time after the pressing is finished; a conversion step, performing a mathematical function conversion of the above relative displacement with respect to a spatial contour at each observation time point, so as to respectively obtain one or more order vibrational modes representing the contour line of the time points; and a determining step, respectively comparing the one or more order vibrational modes of the cornea of the target subject with a corresponding order vibrational mode of at least one reference cornea, and determining a morphology of the corneal topography of the cornea of the target subject.

### BACKGROUND

### Technical Field

The present invention relates to a morphology determining method and a morphology determining system of corneal topography. Specifically, the present invention relates to a morphology determining method and a morphology determining system of corneal topography for performing evaluation by using changes of a contour line of a cornea.

### Related Art

Corneas are important components of eyes. If morphologies of the corneas are abnormal, a problem, for example, myopia, hyperopia, presbyopia, or astigmatism may be caused. In addition, if the corneas are excessively thin or have steep radians that are excessively depressed or protruded, corneal deformation or rupture may also additionally occur to further cause severe eye diseases, for example, ametropia, blurred vision, visual distortion, visual shadows, corneal ectasia, keratoconus, corneal detachment, and blindness.

Furthermore, in some cases, even though keratopathy or abnormality does not occur in corneas in some morphologies, the corneas in such morphologies may still be unsuitable for receiving a specific ophthalmic operation or eye treatment. Therefore, before the ophthalmic operation or eye treatment is performed, the morphologies of the corneas need to be determined in advance to decrease or avoid possible damage to the eyes. However, thicknesses of the corneas are very small, and therefore, it is difficult to detect slight keratopathy or corneal abnormality in an early stage, or learn or determine morphologies of the corneas in an early stage. In addition, inference and determining usually need to be performed by using a special instrument, such as, a corneal topographer, or depending on an experienced medical laboratory scientist based on detection of a low level instrument, such as, a slit lamp. As a result, time costs are increased, and diagnosis and treatment is delayed, which is consequently not beneficial to popularization of detection and application of corneal morphologies.

### SUMMARY

Technical means for resolving problems

To resolve the above problems, an embodiment of the present invention provides a morphology determining method of corneal topography, including the following steps: a parameter obtaining step, based on an original contour line of a cornea of a target subject as the reference, obtaining a relative displacement of each observation point of a contour line of the pressed cornea changed with time from the beginning of pressing the cornea by an external pressure to a predetermined time after the pressing is finished; a conversion step, performing a mathematical function conversion of the relative displacement of the each observation point of the contour line of the cornea changed with time with respect to a spatial contour at each observation time point, so as to respectively obtain one or more order vibrational modes representing the contour line of the time points; and a determining step, respectively comparing the one or more order vibrational modes of the cornea of the target subject with a corresponding order vibrational mode of at least one reference cornea, and determining a morphology of the corneal topography of the cornea of the target subject.

Another embodiment of the present invention provides a morphology determining system of corneal topography, including: a tonometer, a corneal data acquisition module, and a calculating module. The tonometer is configured to exert an external pressure on a cornea of a target subject. The corneal data acquisition module is configured to detect at least one parameter or obtain data capable of calculating the at least one parameter through conversion. The at least one parameter is, based on an original contour line of the cornea as the reference, a relative displacement of each observation point of a contour line of the pressed cornea changed with time from the beginning of pressing the cornea by an external pressure to a predetermined time after the pressing is finished. The calculating module is configured to perform: a parameter obtaining step, obtaining at least one parameter from the corneal data acquisition module; and a conversion step, performing a mathematical function conversion of the at least one parameter with respect to a spatial contour at each observation time point, so as to respectively obtain one or more order vibrational modes representing the contour line of the time points.

Technical effect as compared with the prior art

According to the morphology determining method and the morphology determining system of corneal topography provided in the embodiments of the present invention, based on the relative displacement of the contour line of the pressed cornea, the one or more order vibrational modes for determining the morphology of the corneal topography may be obtained through conversion. Based on the above, one or more order vibrational modes of different corneas may be analyzed and compared based on a computer or manually, so as to obtain information related corneal morphology of a specific target subject more accurately and easily. Therefore, the morphology determining method and the morphology determining system may be used more widely for evaluating and determining the corneal morphology or corneal features of the specific target subject, and a detection probability of corneal abnormality or keratopathy may be further improved.

### BRIEF DESCRIPTION OF THE DRAWINGS

FIG. 1 is a flowchart of a morphology determining method of corneal topography according to an embodiment of the present invention;

FIG. 2 is a schematic diagram of a morphology determining system of corneal topography according to an embodiment of the present invention;

FIG. 3 is a simple schematic diagram of an eye structure of a target subject according to an embodiment of the present invention;

FIG. 4 and FIG. 5 are schematic diagrams of changes of a contour line obtained by pressing a cornea of a target subj ect according to an embodiment of the present invention;

FIG. 6 is a schematic diagram of a contour line of a pressed cornea changed with time according to an embodiment of the present invention;

FIG. 7 is an exemplary schematic diagram of a possible wobbling or changing mode of a cornea according to embodiments of the present invention;

FIG. 8 is a schematic diagram of a relative displacement of each observation point of a contour line of a pressed cornea at a specific time point according to an embodiment of the present invention;

FIG. 9A and FIG. 9B are schematic diagrams highlighting changes of different parts of a cornea in different order vibrational modes according to an embodiment of the present invention;

FIG. 10 is a curve diagram of corresponding first six order vibrational modes of a pressed standard cornea obtained through conversion by using Legendre transformation formula according to an embodiment of the present invention;

FIG. 11 is a schematic diagram of correcting a relative displacement of a cornea according to a rigid body motion effect according to an embodiment of the present invention;

FIG. 12 is a schematic diagram of an analysis result of a damaged cornea obtained by using a morphology determining method or a morphology determining system according to an embodiment of the present invention, according to an embodiment of the present invention;

FIG. 13 is a schematic diagram of morphology of common types of keratoconus; and

FIG. 14 is a curve diagram of corresponding first six order vibrational modes of a plurality of specific morphological corneas obtained by a parameter obtaining step and a conversion step according to embodiments of the present invention.

### DETAILED DESCRIPTION

Embodiments will be described below, and a person of ordinary skill in the art shall easily understand the spirit and the principle of the present invention with reference to the accompanying drawings. However, even though some specific embodiments may be described in detail in this specification, the embodiments are merely examples, and shall not be regarded as limitative or exhaustive meanings in all aspects. Therefore, for a person of ordinary skill in the art, various variations and amendments of the present invention may be obvious and may be achieved easily without departing from the spirit and the principle of the present invention.

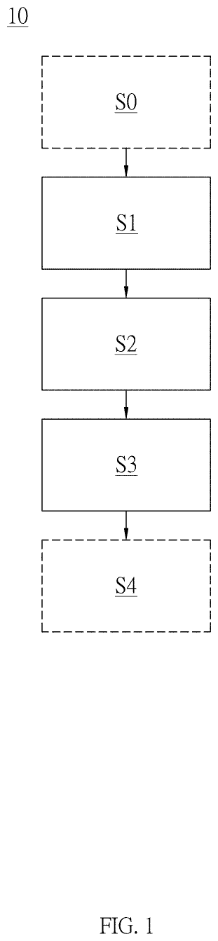

According to an embodiment of the present invention, referring to FIG. 1, a morphology determining method 10 of corneal topography may include the following steps: a parameter obtaining step S1, based on an original contour line of a cornea of a target subject as the reference, obtaining a relative displacement of each observation point of a contour line of the pressed cornea changed with time from the beginning of pressing the cornea by an external pressure to a predetermined time after the pressing is finished; a conversion step S2, performing a mathematical function conversion of the relative displacement of the each observation point of the contour line of the cornea changed with time with respect to a spatial contour at each observation time point, so as to respectively obtain one or more order vibrational modes representing the contour line of the time points; and a determining step S3, respectively comparing the one or more order vibrational modes of the cornea of the target subject with a corresponding order vibrational mode of at least one reference cornea, and determining a morphology of the corneal topography of the cornea of the target subject. In the following, the foregoing steps are further described in detail with reference to other drawings.

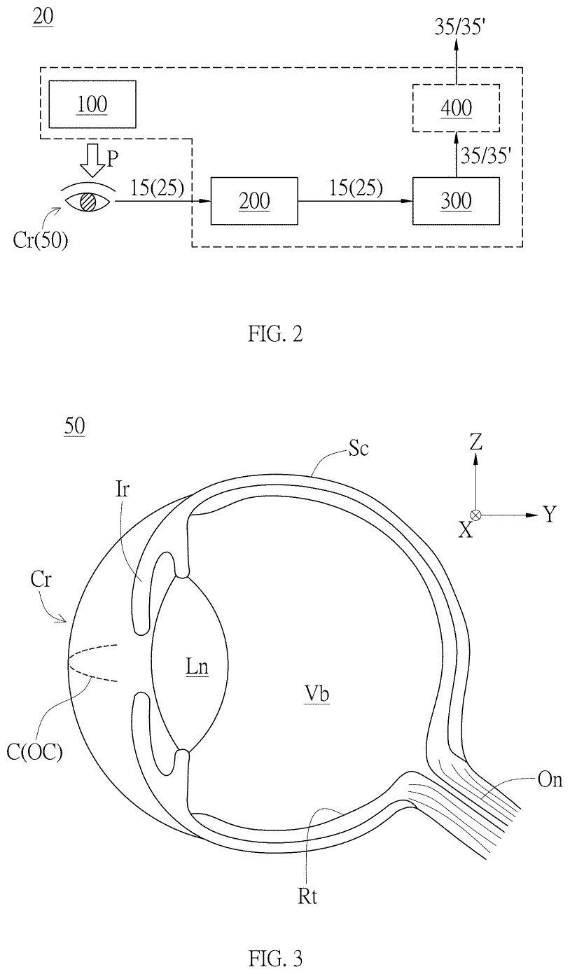

Based on the above, referring to FIG. 2 in conjunction with FIG. 1, according to an embodiment of the present invention, the foregoing morphology determining method 10 may be at least partially or completely performed by using a morphology determining system 20 of corneal topography. Specifically, the morphology determining system 20 may include a tonometer 100, a corneal data acquisition module 200, and a calculating module 300. In addition, in some embodiments, the morphology determining system 20 may further selectively include a display module 400 configured to display an analysis result calculated by the calculating module 300, but the present invention is not limited thereto.

Specifically, refer to FIG. 3 showing an eye structure of a specific target subject 50. The eye structure of the target subject 50 may include components, for example, a cornea Cr, an iris Ir, a sclera Sc, a lens Ln, a vitreous body Vb, a retina Rt, and an optic nerve On, and is roughly the same as or similar to a known eye structure, and therefore, details are not described herein. Based on the above, the eye structure has the cornea Cr facing outwards in a visual direction, and a contour line C may be defined according to a contour of the cornea Cr. Specifically, the contour line C may be a virtual line that intersects with the cornea Cr and that is depicted parallel to a surface of the cornea Cr along an entire contour of the cornea Cr. That is, the contour line C is a contour of a surface of the cornea Cr facing outwards in a specific direction, such as, a contour in an X direction. In this case, as shown in FIG. 3, without any setting, the cornea Cr of the eye structure of the target subject 50 may have an original contour line OC.

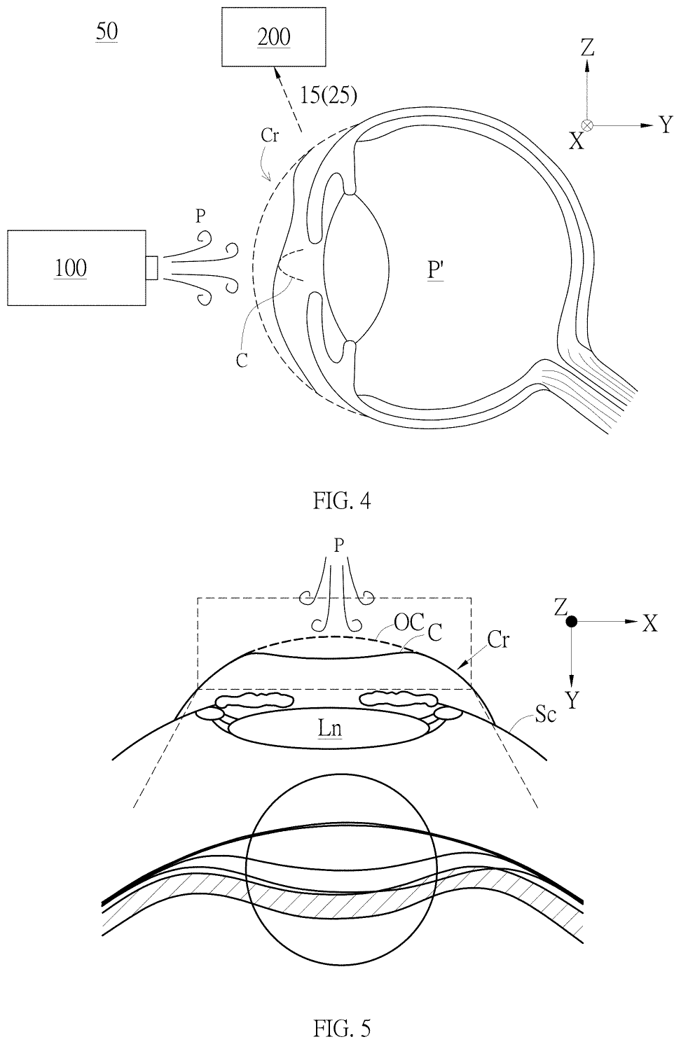

Based on the above, according to an embodiment of the present invention, further referring to FIG. 4 (a schematic side view of an eye) and FIG. 5 (a schematic top view of an eye) in conjunction with FIG. 2 and FIG. 3, the tonometer 100 may be configured to exert an external pressure P on the cornea Cr of the target subject 50. For example, the tonometer 100 may be an air-jet device and may jet air towards the cornea Cr to exert the external pressure P, but in the present invention, the device configured to exert the external pressure P is not limited thereto. Specifically, because of an intraocular pressure P′ inside the eye, when the cornea Cr is pressed by the external pressure P, the cornea Cr may generate corresponding deformation because of the external pressure P and the intraocular pressure P′. When the external pressure P is exerted and after the pressing is finished, the cornea Cr may rebound because of the intraocular pressure P′ to correspondingly generate different deformation morphology of the cornea Cr changed with time. However, factors causing deformation of the pressed cornea Cr with time are not limited thereto, and morphology of the cornea Cr may affect levels, forms and duration of the deformation. For example, when the cornea Cr is relatively thin, the deformation level may be relatively large, and a reaction time from the pressing to the deformation may be relatively short. Then, the corneal data acquisition module 200 may be configured to detect at least one parameter 25 or obtain data 15 capable of calculating the at least one parameter 25 through conversion. The at least one parameter 25 is, based on an original contour line OC of a cornea Cr as the reference, a relative displacement of each observation point of a contour line C of the pressed cornea Cr changed with time from the beginning of pressing the cornea Cr by an external pressure P to a predetermined time after the pressing is finished.

A process implemented by the foregoing corneal data acquisition module 200 may be a data acquisition step S0 implemented before the parameter obtaining step S1 in the morphology determining method 10 (as shown in FIG. 1). For example, the corneal data acquisition module 200 may include a high-speed camera. In the data acquisition step S0, the high-speed camera may take an image group (data 15) of the target subject 50, and then the image group is analyzed in the corneal data acquisition module 200 or the calculating module 300 (for example, the calculating module 300 can obtain and parse the image group (for example, image processing is performed), so as to obtain the at least one parameter). Therefore, changes of the contour line C of the cornea Cr may be obtained. Specifically, the image group presents a series of changes over time of the cornea Cr of the target subject 50 from the beginning of pressing the cornea Cr by an external pressure P to the predetermined time after the pressing is finished. Based on the above, although the at least one parameter 25 (a relative displacement in a digital form) is not directly recorded, the image group includes information related to the at least one parameter 25, and may be additionally parsed and analyzed to obtain the at least one parameter 25 (a relative displacement in a digital form).

Next, the calculating module 300 may implement the parameter obtaining step S1 to obtain the foregoing at least one parameter 25, and then correspondingly implement the conversion step S2. For example, in the parameter obtaining step S1, the calculating module 300 may parse the foregoing image group to obtain the relative displacement (that is, the at least one parameter 25) of each observation point of the contour line C of the pressed cornea Cr changed with time. Alternatively, the image group may be parsed through the corneal data acquisition module 200 or another component, and in the parameter obtaining step S1, the calculating module 300 may directly obtain the relative displacement (that is, the at least one parameter 25) of each observation point of the contour line C of the pressed cornea Cr changed with time from the corneal data acquisition module 200.

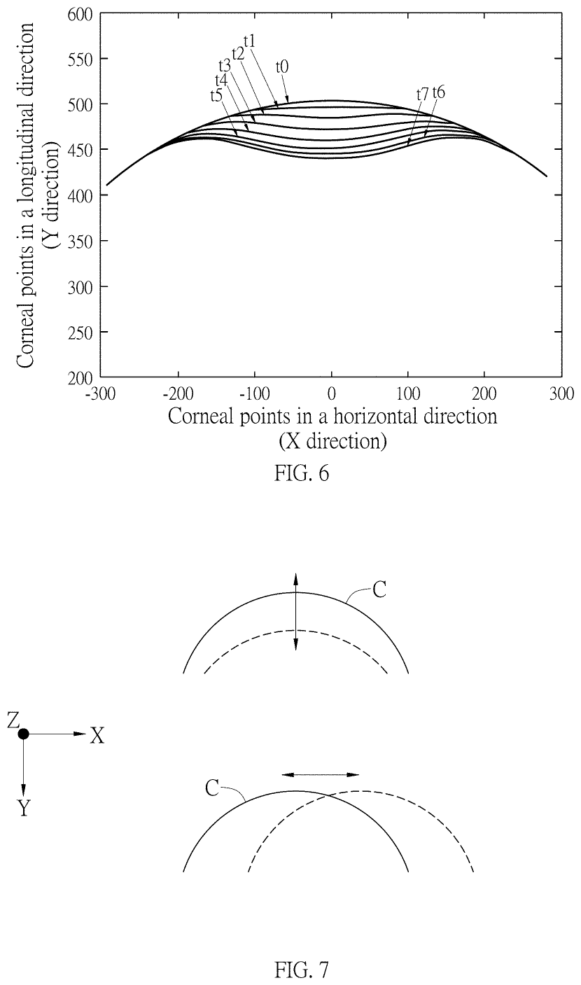

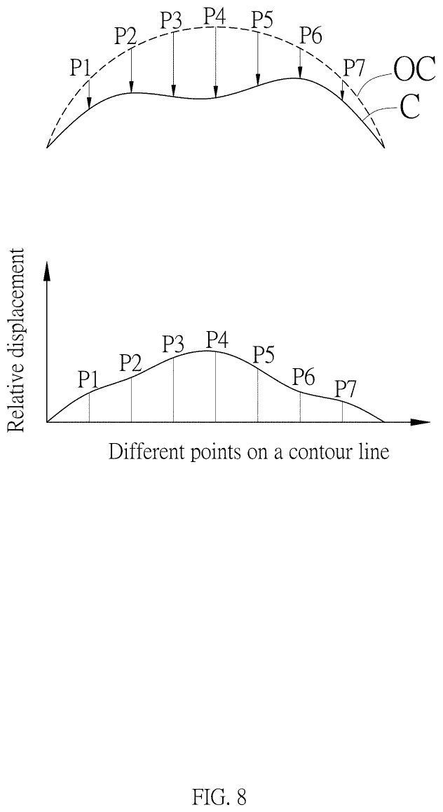

As described above, corresponding to the conversion step S2 in the morphology determining method 10, the calculating module 300 may perform a mathematical function conversion of the at least one parameter 25 with respect to a spatial contour at each observation time point, so as to respectively obtain one or more order vibrational modes 35 representing the contour line C of the cornea Cr of the time points. Specifically, referring to FIG. 6 in conjunction with FIG. 1 to FIG. 5, because of pressing and restoring to an original state naturally, the contour line C of the cornea Cr is changed with time, and has different shapes at different time points, for example, time points t0, t1, t2, t3, t4, t5, t6, and t7 (for example, the time point t0 may be a moment just before the cornea Cr is pressed). In addition, changes of the cornea Cr in processes of wobbling under pressing and restoring to an original state naturally may also be different with different properties of the cornea Cr (for example, thicknesses, pathological phenomena, components, and shapes). For example, as shown in FIG. 7, an even cornea Cr may only generate depression and rebound wobbling as shown in an upper half of FIG. 7 but may also generate left-and-right shaking and wobbling due to uneven cornea Cr thicknesses of two left and right halves of the cornea Cr of an eye. As indicated above, the foregoing description is only an example, wobbling and changes of the cornea Cr that may occur according to different embodiments of the present invention are not limited thereto, and the conversion step S2 of converting the changes of the contour line C of the cornea Cr into specific data is further described in detail in the following.

According to this embodiment, as shown in FIG. 8, at each time point (for example, a time point t1), each observation point (for example, observation points P1, P2, P3, P4, P5, P6, and P7) of a contour line C of a pressed cornea Cr has a relative displacement from a position of a previous original contour line OC. Therefore, based on the concept, the relative displacement (that is, at least one parameter 25) of the each observation point of the contour line C of the pressed cornea Cr changed with time may be then, for example, converted with respect to a spatial contour at each observation time point (for example, time points t0, t1, t2, t3, t4, t5, t6, and t7) by using a calculating module 300, so as to obtain one or more order vibrational modes 35.

It should be noted that, the observation points P1, P2, P3, P4, P5, P6, and P7 are only examples, and according to this embodiment, the core of the concept of performing detection through calculation is actually an entire contour line C. Therefore, a quantity of the observation points that should be selected may be decided according to a computing capability and a result accuracy requirement.

Specifically, the mathematical function conversion with respect to the spatial contour at the each observation time point of the contour line C may be performed, for example, by using Legendre transformation formula or Fourier transformation formulas, and may be implemented, for example, by using the calculating module 300 of the morphology determining system 20, but the present invention is not limited thereto.

Specifically, for example, the conversion with respect to the spatial contour at the each observation time point may be performed by using the following Legendre transformation formula (Formula 1) based on at least one parameter 25:

$${a}_{n} ( t ) = \frac{2 n + 1}{2} {\int }_{0}^{π} f ( θ , t ) {P}_{n} ( cos θ ) sin θdθ \qquad Fomula 1$$

where aₙ(t) is a value of Legendre coefficient changed with time, n is an order value of different order vibrational modes, and f(θ,t) is a value of the relative displacement at different angles, where the different angles refer to a coordinate angle of the each observation point, Pₙ(cos θ) is Legendre polynomial, and the coordinate angle of the each observation point is θ.

Alternatively, for example, the conversion with respect to the spatial contour at the each observation time point may be performed by using the following Fourier transformation formulas (Formula 2 to Formula 4) based on at least one parameter 25:

$$\begin{matrix}{a}_{0} ( t ) = \frac{2}{π} {\int }_{0}^{π} f ( θ , t ) d θ & Fomula 2 \\ {a}_{n} ( t ) = \frac{2}{π} {\int }_{0}^{π} f ( θ , t ) cos ( 2 n θ ) d θ & Fomula 3 \\ {b}_{n} ( t ) = \frac{2}{π} {\int }_{0}^{π} f ( θ , t ) sin ( 2 n θ ) d θ & Fomula 4\end{matrix}$$

where a₀(t) is a value of Fourier coefficient applicable to a zeroth mode changed with time, aₙ(t) and bₙ(t) are respectively values of Fourier coefficients applicable to even order vibrational modes and odd order vibrational modes changed with time, and f(θ, t) is a value of the relative displacement at different angles, where the different angles refer to a coordinate angle of the each observation point, and the coordinate angle of the each observation point is θ.

Based on the above, in the conversion step S2, by using the Legendre transformation formula or the Fourier transformation formulas, one or more order vibrational modes 35 representing the contour line C of the cornea Cr of the time points are respectively obtained. For example, in an embodiment, the one or more order vibrational modes 35 may include at least first six order vibrational modes: a zeroth mode M0 (n=0), a first mode M1 (n=1), a second mode M2 (n=2), a third mode M3 (n=3), a fourth mode M4 (n=4), and a fifth mode M5 (n=5). However, the above is only an example, and a higher order vibrational mode (higher than the fifth mode) may be calculated through conversion, and according to the present invention, a quantity of the one or more order vibrational modes 35 is not limited to the first six order modes in this example.

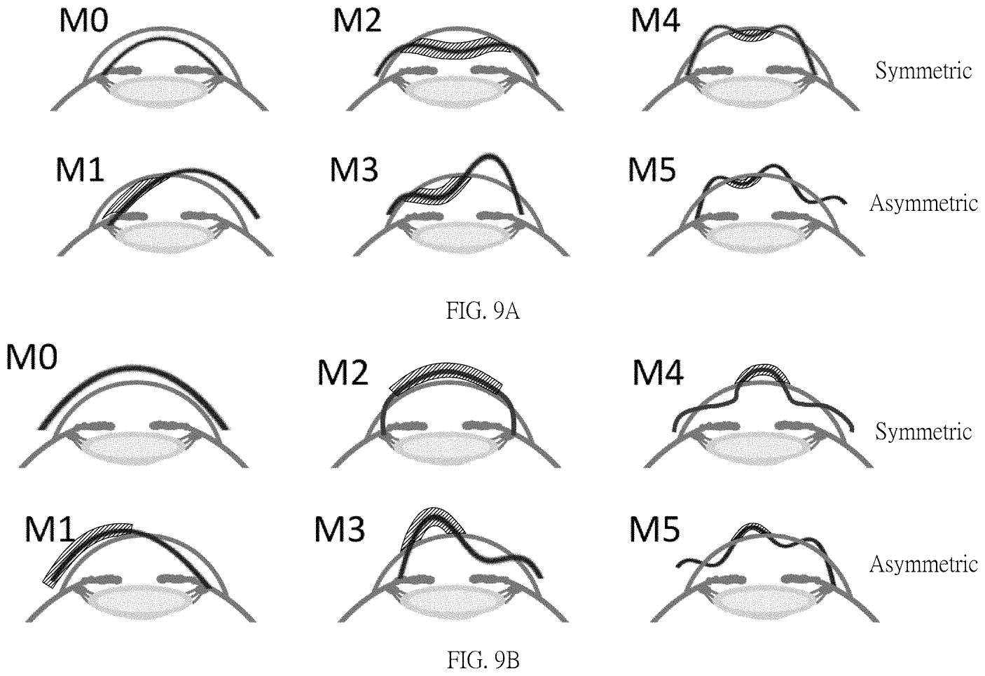

According to an embodiment of the present invention, definitions of being positive and negative in the foregoing conversion step S2 may be as shown in FIG. 9A and FIG. 9B. As mentioned above, to calculate the relative displacement, corresponding to each order vibrational mode, corneal changes in a positive displacement direction may be, for example, defined as shown in FIG. 9A, and corresponding to each order vibrational mode, corneal changes in a negative displacement direction may be, for example, defined as shown in FIG. 9B. Therefore, curve diagrams of the one or more order vibrational modes 35 may be obtained through conversion in the conversion step S2, and therefore, features of specific parts of the cornea Cr may be highlighted and reflected.

Specifically, according to this embodiment, after the conversion with respect to the spatial contour at the each observation time point is performed, for example, after the conversion is performed by using the Legendre transformation formula or the Fourier transformation formulas, the zeroth mode M0 may highlight a feature of moving backward of an entire shape of the cornea Cr, the first mode M1 may highlight a feature of rotating left and right of a shape of a ½ part of the cornea Cr, the second mode M2 may highlight a feature of depression of a shape of a ⅓ part in a center of the cornea Cr, the third mode M3 may highlight a feature of high-and-low distortion of a shape of a ¼ part adjacent to the center of the cornea Cr, the fourth mode M4 may highlight a feature of depression of a shape of a ⅕ part in the center of the cornea Cr, and the fifth mode (M5) may highlight a feature of high-and-low distortion of a shape of a ⅙ part adjacent to the center of the cornea Cr. Therefore, the curve diagrams, obtained through conversion, of the one or more order vibrational modes 35 of the cornea Cr of the specific target subject 50 may be then used in the following determining step S3. For example, the one or more order vibrational modes 35 of the cornea Cr of the target subject 50 are respectively compared with corresponding order vibrational modes of at least one reference cornea by a calculating module 300 or a related person, and therefore, a morphology of corneal topography of the cornea Cr of the target subject 50 may be determined and evaluated.

Based on the above, referring to FIG. 9A and FIG. 9B, parts highlighted by each order vibrational mode are particularly presented in bold for emphasis. Based on an original contour line OC of the cornea Cr, for the zeroth mode M0, a relative displacement of an entire shape of the cornea Cr depressed inwards may be defined as a positive value; for the first mode M1, a relative displacement of a left ½ part adjacent to a center of the cornea Cr depressed inwards may be defined as a positive value; for the second mode M2, a relative displacement of a ⅓ part of the center of the cornea Cr depressed inwards is defined as a positive value; for the third mode M3, a relative displacement of a left ¼ part adjacent to the center of the cornea Cr depressed inwards is defined as a positive value; for the fourth mode M4, a relative displacement of a ⅕ part of the center of the cornea Cr depressed inwards is defined as a positive value; and for the fifth mode M5, a relative displacement of a left ⅙ part adjacent to the center of the cornea Cr depressed inwards is defined as a positive value. Therefore, the one or more order vibrational modes of the cornea Cr of the target subject 50 and the corresponding order vibrational mode of the at least one reference cornea may be calculated.

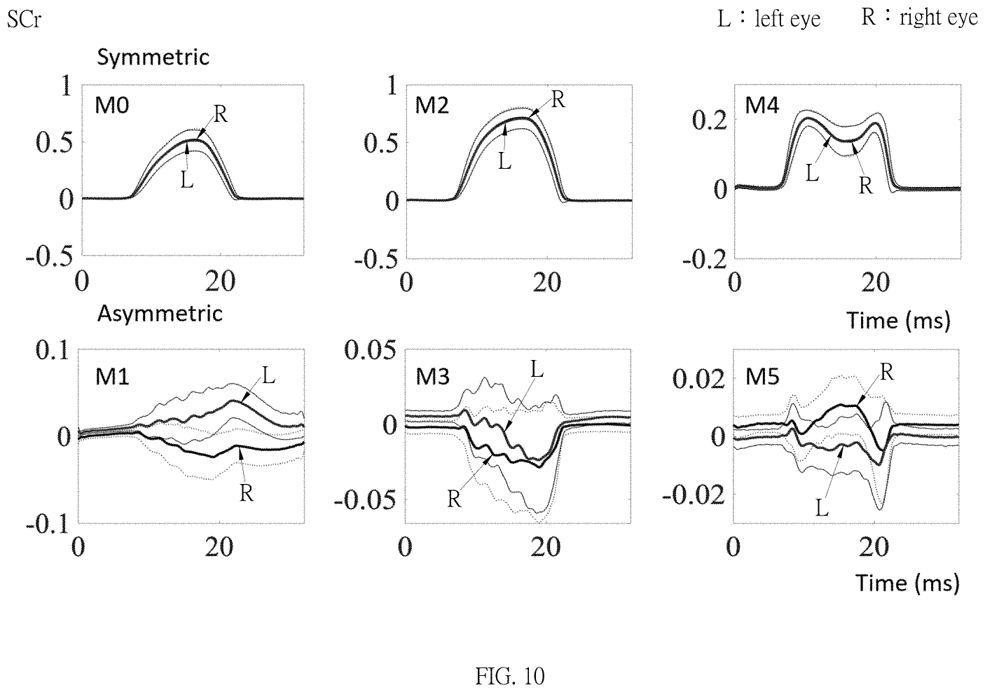

Based on the above, in the foregoing determining step S3 (for example, additionally performed by using the calculating module 300 or manually), the at least one reference cornea compared with the cornea Cr of the current specific target subject 50 may include a standard cornea SCr. Specifically, a corresponding order vibrational mode of the standard cornea SCr may be an average result calculated and established according to normal corneas of a plurality of normal persons substantially without eye diseases based on the parameter obtaining step S1 and the conversion step S2 with a same or similar concept, and can be stored, for example, in the calculating module 300 or is accessible to the calculating module 300. For example, referring to FIG. 10, curve diagrams of corresponding first six order vibrational modes of an average value of a standard cornea SCr are shown as examples. Corresponding background section defined by corresponding line pair represent intervals of standard deviations.

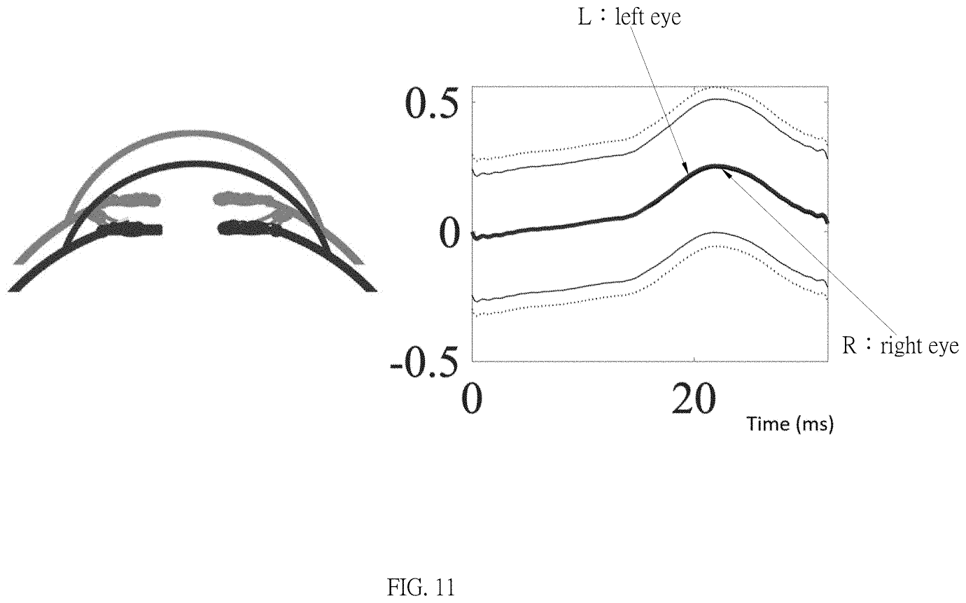

In addition, in some embodiments, referring to FIG. 11, to avoid a calculation error caused by a rigid body motion effect, when any one of the parameter obtaining step S1, the conversion step S2, and the determining step S3 is performed, for example, by using a calculating module 300, a tolerance of changes of a contour line C caused by movement of an entire eye (for example, a person moves backward out of fear) rather than deformation of a cornea Cr may be additionally excluded, and details are not described herein.

As stated above, according to differences between one or more order vibrational modes 35, for example, the zeroth mode M0, the first mode M1, the second mode M2, the third mode M3, the fourth mode M4, and the fifth mode M5 of the cornea Cr and a corresponding order vibrational mode of the standard cornea SCr, the determining step S3 may include respectively determining corneal features of the cornea Cr with respect to the standard cornea SCr based on the corneal features represented by the one or more order vibrational modes, for example, the zeroth mode M0, the first mode M1, the second mode M2, the third mode M3, the fourth mode M4, and the fifth mode M5. Therefore, morphological data 35′ of corneal topography of the cornea Cr may be calculated and evaluated. That is, if an entirety or a local region of the cornea Cr is more uneven, protruded or depressed, or there are other abnormalities or special feature differences, a deviation difference between one or more order vibrational modes 35 of a cornea Cr of a target subject 50 and a corresponding order vibrational mode of a standard cornea SCr is larger. Therefore, the morphological data 35′ of the corneal topography of the cornea Cr may be determined and analyzed by the compared deviation difference.

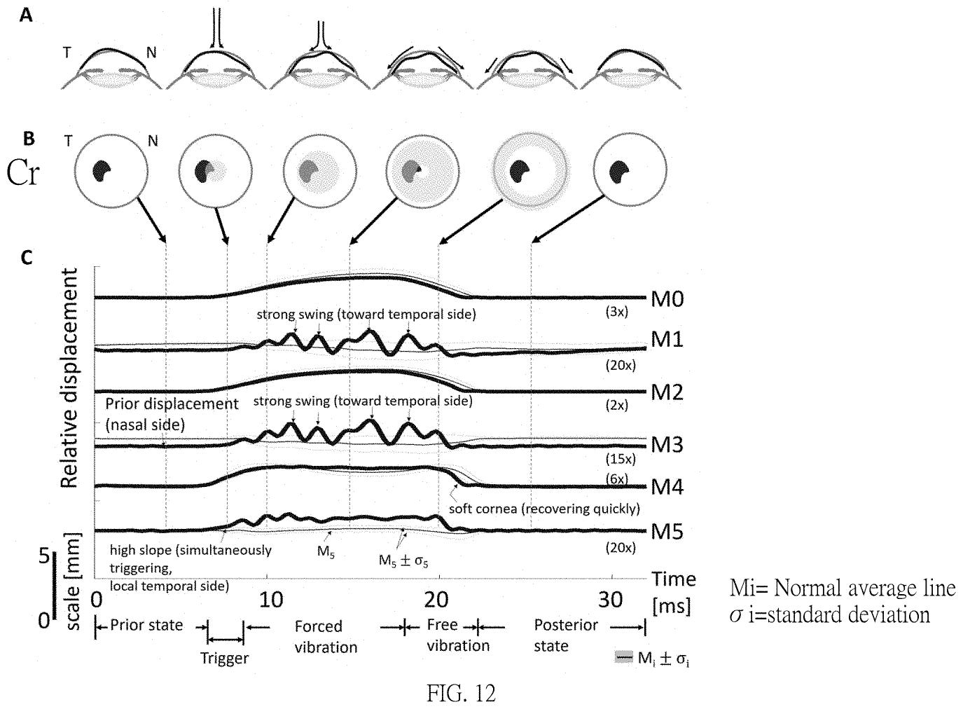

For example, referring to FIG. 10, if there is a cornea Cr of a patient (a target subject 50), a curve in the first mode M1 with respect to a standard cornea SCr has a larger positive value. It may be correspondingly estimated that compared with the standard cornea SCr, a left half of the cornea Cr is thinner, so that the cornea Cr after being pressed is easier to wobble to the right. Furthermore, according to an embodiment, for example, referring to FIG. 12, when a cornea Cr is damaged or thinned in a black region indicated in part B of FIG. 12, performances with time of different order vibrational modes after mode decomposition may reflect corresponding differences very obviously. Specifically, compared with a normal average value (marked with a light line in part C of FIG. 12) of a standard cornea SCr, a damaged cornea Cr shown in part C of FIG. 12 has obvious differences in a first mode M1 (highlighting a shape of a ½ part of the cornea Cr), a third mode M3 (highlighting a ¼ part adjacent to a center of the cornea Cr), and a fifth mode M5 (highlighting a ⅙ part adjacent to the center of the cornea Cr). Therefore, a block in which the cornea Cr is damaged or thinned or abnormal and a corresponding characteristic may be determined from the vibrational modes presenting a difference and a feature (for example, a feature of strong swinging) by using each vibrational mode corresponding to a specific highlighted corneal block. Based on the above, by comparing the deviation differences between the one or more order vibrational modes 35 of the cornea Cr of the target subject 50 and the corresponding order vibrational mode of the standard cornea SCr, a possible damaged or thinned region of the cornea may be quickly defined or derived.

Based on a determining result of the corneal features (morphological data 35′), if the cornea Cr of the target subject 50 has a phenomenon of being relatively thin locally and protruding outwards or being depressed inwards with respect to the standard cornea SCr, it may be determined in the determining step S3 that the target subject 50 suffers from keratoconus or corneal ectasia. However, the present invention is not limited thereto, and morphology or characteristics of the cornea Cr of the target subject 50 may be determined in the determining step S3 based on a corresponding analysis result.

In addition, the at least one reference cornea may also include an artificially set virtual cornea. A corresponding order vibrational mode of the artificially set virtual cornea is not calculated and established according to a cornea of a real person but is preset artificially (may be preset with reference to a cornea of a real person or without foundation), and may be, for example, stored in a calculating module 300 or is accessible to the calculating module 300. Based on the above, according to differences between the cornea Cr and the artificially set virtual cornea with respect to one or more order vibrational modes, for example, the zeroth mode M0, the first mode M1, the second mode M2, the third mode M3, the fourth mode M4, and the fifth mode M5 of the cornea Cr, the determining step S3 may include respectively determining the corneal features of the cornea Cr of the specific target subject 50 with respect to the artificially set virtual cornea based on the corneal features represented by the one or more order vibrational modes, for example, the zeroth mode M0, the first mode M1, the second mode M2, the third mode M3, the fourth mode M4, and the fifth mode M5. The process is similar to the foregoing comparison with respect to the standard cornea SCr, and details are not described herein.

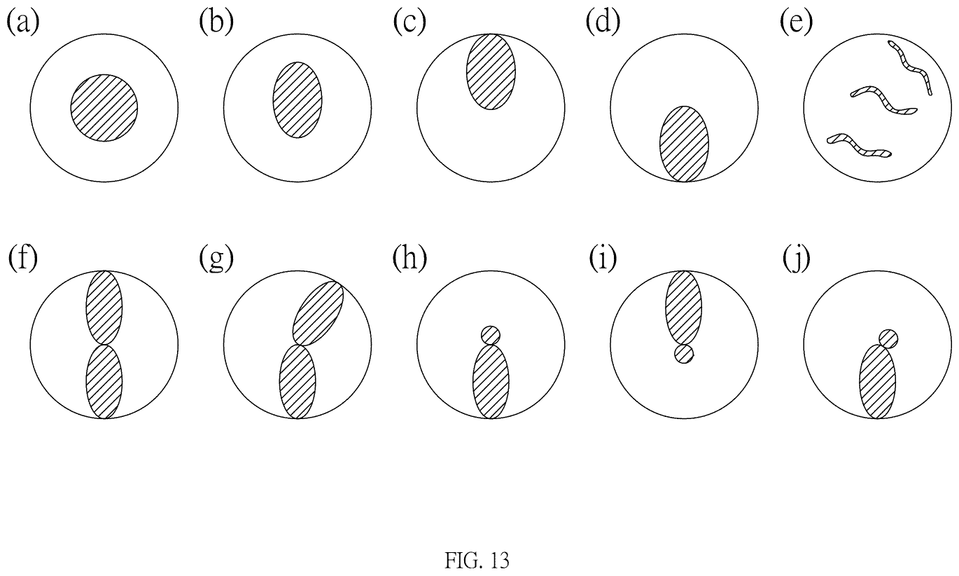

Then, referring to FIG. 13, by using a keratoconus as an example, common abnormal keratopathies are as follows: Sequentially from left to right, and then from top to bottom, parts (a) to (j) represents keratoconus of: a central round, a central oval, a first half superior steepening (SS), a second half inferior steepening (IS), an irregularly-shaped, a symmetric bowtie, a symmetric bowtie with skewed radial axes, a second half asymmetric bowtie with inferior steepening (AB/IS), a first half asymmetric bowtie with superior steepening (AB/SS), and an asymmetric bowtie with skewed radial axes. However, the above are only examples of common keratoconus, and morphology types of corneas are not limited thereto.

Based on the above, according to the morphology determining method 10 and the morphology determining system 20, if a cornea is divided into a first half and a second half, positions in the first half and the second half in which a phenomenon that the cornea is relatively thin or protruded occurs may be determined according to differences between one or more order vibrational modes 35 of a cornea Cr of a target subject 50 and a corresponding order vibrational mode of at least one reference cornea (for example, a standard cornea and/or an artificially set virtual cornea). Therefore, a corneal morphology type or a corneal characteristic that the cornea Cr of the target subject 50 may have may be evaluated and derived.

For example, compared with a standard cornea and/or an artificially set virtual cornea, if the phenomenon that a cornea is relatively thin or protruded does not occur in the cornea Cr of the target subject 50, it may be determined that the target subject 50 has a normal cornea; if the phenomenon that a cornea is relatively thin or protruded is concentrated in a junction between the first half and the second half, it may be determined that the target subject 50 has a central round or central oval keratoconus; if the phenomenon that a cornea is relatively thin or protruded only occurs in one of the first half and the second half, it may be determined that the target subject 50 has a first half superior steepening (SS) keratoconus or a second half inferior steepening (IS) keratoconus ; if phenomena that corneas is relatively thin or protruded respectively occur in the first half and the second half with essentially same sizes, it may be determined that the target subject 50 has a symmetric bowtie keratoconus; if phenomena that corneas is relatively thin or protruded respectively occur in the first half and the second half with essentially different sizes, it may be determined that the target subject 50 has an asymmetric bowtie keratoconus; if an included angle between axes of blocks, of the first half and the second half, in which corneas are relatively thin or protruded is less than 150°, it may be determined that the target subject 50 has a symmetric bowtie with skewed radial axes keratoconus or an asymmetric bowtie with skewed radial axes keratoconus; and if the phenomenon that a cornea is relatively thin or protruded occurs and does not belong to any one of the above items, it may be determined that the target subject 50 has an irregularly-shaped keratoconus. However, it should be noted that, the foregoing descriptions are only conceptual examples, and possible corresponding keratopathy or a corneal morphology type may be evaluated and depicted according to a position in which a difference between the cornea Cr of the target subject 50 and the standard cornea and/or the artificially set virtual cornea occurs. Based on the above, in another embodiment, the morphology determining method 10 and the morphology determining system 20 may be used for determining another keratopathy or corneal morphology type other than the keratopathies or the corneal morphology types shown in FIG. 13, but the present invention is not limited thereto.

Furthermore, the at least one reference cornea may also include one or more specific morphological corneas (for example, the various types of the foregoing keratoconus), and respective corresponding order vibrational modes of the one or more specific morphological corneas may be respectively calculated and established according to each of the actual one or more specific morphological corneas based on the parameter obtaining step S1 and the conversion step S2.

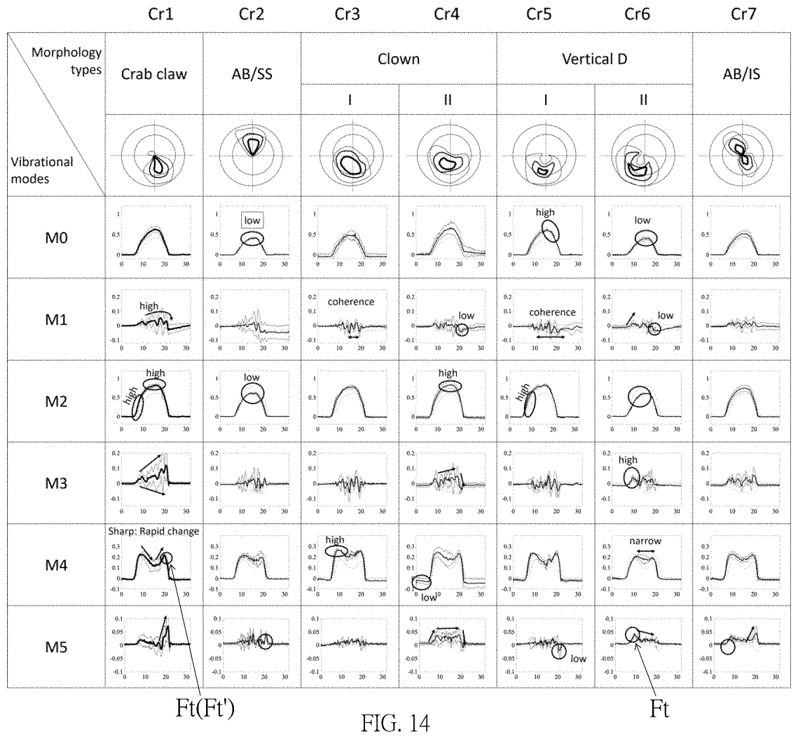

For example, according to an embodiment, corneal topographies of different specific morphological corneas Cr1 to Cr7 depicted by using a corneal topographer are as shown in FIG. 14 (Morphology types). Specifically, increased steepness is indicated and defined with increased line thickness, and a steeper part may represent or suggest a block in which a cornea may be relatively thin or a keratopathy or an abnormality occurs. Based on the above, according to an embodiment of the present invention, for each of the morphological corneas Cr1 to Cr7, corresponding order vibrational modes (for example, corresponding first six order vibrational modes) calculated and established by using the parameter obtaining step S1 and the conversion step S2 are listed under corneal topographies of the different specific morphological corneas Cr1 to Cr7. Therefore, a morphology of corneal topography of a cornea Cr of a target subject 50 may be determined by pre-building corresponding order vibrational modes of the different specific morphological corneas Cr1 to Cr7 of the existing corneal topography, or by comparing one or more order vibrational modes of a cornea Cr of a target subject 50 with the corresponding order vibrational modes of the one or more specific morphological corneas Cr1 to Cr7.

Specifically, according to some embodiments, at least one corresponding order vibrational mode of different corresponding order vibrational modes of each of the one or more specific morphological corneas Cr1 to Cr7 may have at least one curve feature Ft. For example, a fourth mode (n=4) of the specific morphological cornea Cr1 may have a curve feature Ft′ of a rapid change as shown in an enclosed part. In addition, the corresponding order vibrational modes of the one or more specific morphological corneas Cr1 to Cr7 or the curve features Ft of the one or more specific morphological corneas Cr1 to Cr7 are stored in the calculating module 300 or are accessible to the calculating module 300. Therefore, the determining step S3 may include determining, through comparison, whether the one or more order vibrational modes 35 of the cornea Cr of the target subject 50 have the corresponding curve features in the respective corresponding vibrational modes. For example, the fourth mode (n=4) has the curve feature Ft′ of the rapid change as shown in the enclosed part of the specific morphological cornea Cr1 in FIG. 14, or for example, but is not limited to another curve features highlighted with marks with respect to a reference value shown in FIG. 14. Based on the above, compared with any specific morphological cornea, when the one or more order vibrational modes 35 of the cornea Cr have the curve features above a preset threshold (for example, an entirety, 90%, 80%, or at least a half), it may be determined in the determining step S3 that the cornea Cr is a corresponding morphological cornea of the one or more specific morphological corneas Cr1 to Cr7 having the curve features. For example, the cornea Cr may have a curve feature corresponding to the specific morphological cornea Cr1 in the first mode M1, have a curve feature corresponding to the specific morphological cornea Cr1 in the second mode M2, have a curve feature corresponding to the specific morphological cornea Cr1 in the third mode M3, have a curve feature corresponding to the specific morphological cornea Cr1 in the fourth mode M4, and have a curve feature corresponding to the specific morphological cornea Cr1 in the fifth mode M5, and therefore, it may be determined that a morphology of the cornea Cr is a type of the specific morphological cornea Cr1.

In addition, similarly, also referring to FIG. 14, when a quantity of the specific morphological corneas Cr1 to Cr7 that can be compared does not cause a burden, or when the comparison is performed manually or by using a calculating module 300 that performs comparison accurately enough, complete curve forms rather than specific local curve features may also be compared directly. Specifically, the corresponding order vibrational modes of the one or more specific morphological corneas Cr1 to Cr7 are respectively calculated and established substantially according to each of the one or more specific morphological corneas Cr1 to Cr7 based on the parameter obtaining step S1 and the conversion step S2, and are stored in the calculating module 300 or are accessible to the calculating module 300. In the determining step S3, each vibrational mode of the one or more order vibrational modes 35 of the cornea Cr is compared with a corresponding vibrational mode of each of the one or more specific morphological corneas Cr1 to Cr7, and when the one or more order vibrational modes 35 of the cornea Cr accord with curve forms above a preset mode value, the determining step S3 determines that the cornea Cr is a corresponding morphological cornea of the one or more specific morphological corneas Cr1 to Cr7 according with the curve forms. For example, if compared with other specific morphological corneas, curve diagrams of first six order vibrational modes of the cornea Cr of the target subject 50 are each more accordant with or essentially the same as those of all of the corresponding first six order vibrational modes of the specific morphological cornea Cr1, it may be determined that a morphology of the cornea Cr of the target subject 50 is a type of the specific morphological cornea Cr1.

It should be noted that, the specific morphological corneas Cr1 to Cr7 shown in FIG. 14 are only examples, and the types and the quantity of the specific morphological corneas that can be compared with the cornea Cr of the target subject 50 are not limited thereto according to the embodiments of the present invention. That is, a lookup table corresponding to different morphological corneas may be established, and the cornea Cr of the target subject 50 may be compared based on the lookup table in the determining step S3.

To sum up, based on the morphology determining method and the morphology determining system of corneal topography according to the embodiments of the present invention, by using the relative displacement of the contour line of the pressed cornea, the one or more order vibrational modes including information about the morphology of the corneal topography may be obtained through conversion. Therefore, the morphology of the corneal topography of the cornea may be parsed more easily and accurately by using an instrument that can obtain a relative displacement of a contour line of the cornea without using a corneal topographer. Therefore, popularization of application of determining morphology of corneal topography can be facilitated, and corneal abnormality or keratopathy can be detected or diagnosed in advance, so that corresponding proper treatment measures can be taken.

The above are only some exemplary embodiments of the present invention. It should be noted that various variations and modifications can be made to the present invention without departing from the spirit and principle of the present invention. A person of ordinary skill in the art should understand that the present invention is defined by the appended claims, and in accordance with the intent of the present invention, various possible changes, such as substitution, combination, modification, and conversion, all fall within the scope defined by the appended claims of the present invention.

### REFERENCE NUMERALS

Ir: Iris

Sc: Sclera

Ln: Lens

Cr: Cornea

SCr: Standard cornea

Cr1-Cr7: Morphological cornea

C: Contour line

OC: Original contour line

Vb: Vitreous body

On: Optic nerve

Rt: Retina

Ft, Ft′: Curve feature

10: Morphology determining method

15: Data

20: Morphology determining system

25: Parameter

35: Mode

35′: Morphological data

50: Target subject

100: Tonometer

200: Corneal data acquisition module

300: Calculating module

400: Display module

P: External pressure

P′: Intraocular pressure

S0: Data acquisition step

S1: Parameter obtaining step

S2: Conversion step

S3: Determining step

S4: Display step

t0, t1, t2, t3, t4, t5, t6, and t7: Time point

P0, P1, P2, P3, P4, P5, P6, and P7: Observation point

M0: Zeroth mode

M1: First mode

M2: Second mode

M3: Third mode

M4: Fourth mode

M5: Fifth mode

What is claimed is:

1. A morphology determining method of corneal topography, comprising the following steps:
a parameter obtaining step (S1), based on an original contour line of a cornea of a target subject as the reference, obtaining a relative displacement of each observation point of a contour line of the pressed cornea changed with time from the beginning of pressing the cornea by an external pressure to a predetermined time after the pressing is finished;
a conversion step (S2), performing a mathematical function conversion of the relative displacement of the each observation point of the contour line of the cornea changed with time with respect to a spatial contour at each observation time point, so as to respectively obtain one or more order vibrational modes representing the contour line of the time points; and
a determining step (S3), respectively comparing the one or more order vibrational modes of the cornea of the target subject with a corresponding order vibrational mode of at least one reference cornea, and determining a morphology of the corneal topography of the cornea of the target subject.

2. The morphology determining method of corneal topography according to claim 1, further comprising:
a data acquisition step (S0), implemented before the parameter obtaining step (S1), to obtain and parse an image group of the target subject, so as to obtain changes of the contour line of the cornea, wherein the image group presents a series of changes over time of the cornea of the target subject from the beginning of pressing the cornea by the external pressure to the predetermined time after the pressing is finished, and
the parameter obtaining step (S1) obtaining the relative displacement of the each observation point of the contour line of the pressed cornea changed with time by parsing the image group.

3. The morphology determining method of corneal topography according to claim 1, wherein
the mathematical function conversion with respect to the spatial contour at the each observation time point is performed by using the following Legendre transformation formula,
$${a}_{n} ( t ) = \frac{2 n + 1}{2} {\int }_{0}^{π} f ( θ , t ) {P}_{n} ( cos θ ) sin θdθ$$
wherein aₙ(t) is a value of Legendre coefficient changed with time, n is an order value of different order vibrational modes, and f(θ, t) is a value of the relative displacement at different angles, wherein the different angles refer to a coordinate angle of the each observation point, Pₙ(cos θ) is Legendre polynomial, and the coordinate angle of the each observation point is θ.

4. The morphology determining method of corneal topography according to claim 1, wherein
the mathematical function conversion with respect to the spatial contour at the each observation time point is performed by using the following Fourier transformation formulas,
$${a}_{0} ( t ) = \frac{2}{π} {\int }_{0}^{π} f ( θ , t ) d θ$$
$${a}_{n} ( t ) = \frac{2}{π} {\int }_{0}^{π} f ( θ , t ) cos ( 2 n θ ) d θ$$
$${b}_{n} ( t ) = \frac{2}{π} {\int }_{0}^{π} f ( θ , t ) sin ( 2 n θ ) d θ$$
wherein a₀(t) is a value of Fourier coefficient applicable to a zeroth mode changed with time, aₙ(t) and bₙ(t) are respectively values of Fourier coefficients applicable to even order vibrational modes and odd order vibrational modes changed with time, and f(θ, t) is a value of the relative displacement at different angles, wherein the different angles refer to a coordinate angle of the each observation point, and the coordinate angle of the each observation point is θ.

5. The morphology determining method of corneal topography according to claim 1, wherein the one or more order vibrational modes comprise at least first six order vibrational modes: a zeroth mode (M0), a first mode (M1), a second mode (M2), a third mode (M3), a fourth mode (M4), and a fifth mode (M5), wherein
the zeroth mode (M0) highlights a feature of moving backward of an entire shape of the cornea, the first mode (M1) highlights a feature of rotating left and right of a shape of a ½ part of the cornea, the second mode (M2) highlights a feature of depression of a shape of a ⅓ part in a center of the cornea, the third mode (M3) highlights a feature of high-and-low distortion of a shape of a ¼ part adjacent to the center of the cornea, the fourth mode (M4) highlights a feature of depression of a shape of a ⅕ part in the center of the cornea, and the fifth mode (M5) highlights a feature of high-and-low distortion of a shape of a ⅙ part adjacent to the center of the cornea.

6. The morphology determining method of corneal topography according to claim 5, wherein the at least one reference cornea comprises a standard cornea, and a corresponding order vibrational mode of the standard cornea is calculated and established substantially according to normal corneas of a plurality of normal persons without eye diseases based on the parameter obtaining step (S1) and the conversion step (S2), and according to differences between the cornea and the standard cornea with respect to the zeroth mode (M0), the first mode (M1), the second mode (M2), the third mode (M3), the fourth mode (M4), and the fifth mode (M5), the determining step (S3) comprises respectively determining corneal features of the cornea with respect to the standard cornea based on the corneal features represented by the zeroth mode (M0), the first mode (M1), the second mode (M2), the third mode (M3), the fourth mode (M4), and the fifth mode (M5).

7. The morphology determining method of corneal topography according to claim 6, wherein based on a determining result of the corneal features, if the cornea of the target subject has a phenomenon of being relatively thin locally and protruding outwards or being depressed inwards with respect to the standard cornea, it is determined in the determining step (S3) that the target subject suffers from keratoconus or corneal ectasia.

8. The morphology determining method of corneal topography according to claim 5, wherein the at least one reference cornea comprises an artificially set virtual cornea, and a corresponding order vibrational mode of the artificially set virtual cornea is preset, and according to differences between the cornea and the artificially set virtual cornea with respect to the zeroth mode (M0), the first mode (M1), the second mode (M2), the third mode (M3), the fourth mode (M4), and the fifth mode (M5), the determining step (S3) comprises respectively determining corneal features of the cornea with respect to the artificially set virtual cornea based on the corneal features represented by the zeroth mode (M0), the first mode (M1), the second mode (M2), the third mode (M3), the fourth mode (M4), and the fifth mode (M5).

9. The morphology determining method of corneal topography according to claim 1, wherein the at least one reference cornea comprises one or more specific morphological corneas, and respective corresponding order vibrational modes of the one or more specific morphological corneas are respectively calculated and established substantially according to each of the one or more specific morphological corneas based on the parameter obtaining step (S1) and the conversion step (S2).

10. The morphology determining method of corneal topography according to claim 9, wherein at least one corresponding order vibrational mode of different corresponding order vibrational modes of each of the one or more specific morphological corneas has at least one curve feature, and the determining step (S3) comprises determining, through comparison, whether the one or more order vibrational modes of the cornea have the corresponding curve features in the respective corresponding vibrational modes, and
when the one or more order vibrational modes of the cornea have the curve features above a preset threshold, the determining step (S3) determines that the cornea is a corresponding morphological cornea of the one or more specific morphological corneas having the curve features.

11. The morphology determining method of corneal topography according to claim 9, wherein in the determining step (S3), each vibrational mode of the cornea is compared with each of the corresponding order vibrational modes of the one or more specific morphological corneas, and when the one or more order vibrational modes of the cornea accord with curve forms above a preset mode quantity, the determining step (S3) determines that the cornea is a corresponding morphological cornea of the one or more specific morphological corneas according with the curve forms.

12. The morphology determining method of corneal topography according to claim 1, wherein the parameter obtaining step (S1) comprises: obtaining the measured relative displacement changed with time of the each observation point of the contour line of the cornea of the target subject pressed by a tonometer.

13. A morphology determining system of corneal topography, comprising:
a tonometer, configured to exert an external pressure on a cornea of a target subject;
a corneal data acquisition module, configured to detect at least one parameter or obtain data capable of calculating the at least one parameter through conversion, wherein at least one parameter is, based on an original contour line of the cornea as the reference, a relative displacement of each observation point of a contour line of the pressed cornea changed with time from the beginning of pressing the cornea by an external pressure to a predetermined time after the pressing is finished; and
a calculating module, configured to perform:
a parameter obtaining step (S1), obtaining at least one parameter from the corneal data acquisition module; and
a conversion step (S2), performing a mathematical function conversion of the at least one parameter with respect to a spatial contour at each observation time point, so as to respectively obtain one or more order vibrational modes representing the contour line of the time points.

14. The morphology determining system of corneal topography according to claim 13, wherein the calculating module is further configured to perform a determining step (S3) after the parameter obtaining step (S1) and the conversion step (S2), wherein
in the determining step (S3), the calculating module respectively compares the one or more order vibrational modes of the cornea of the target subject with a corresponding order vibrational mode of at least one reference cornea, and determines a morphology of the corneal topography of the cornea of the target subject.

15. The morphology determining system of corneal topography according to claim 13, wherein the corneal data acquisition module comprises a high-speed camera, the high-speed camera takes an image group of the target subject, and the calculating module is capable of obtaining and parsing the image group, so as to obtain the at least one parameter, wherein
the image group presents a series of changes over time of the cornea of the target subject from the beginning of pressing the cornea by the external pressure to the predetermined time after the pressing is finished.

16. The morphology determining system of corneal topography according to claim 13, wherein
the calculating module of the morphology determining system performs the mathematical function conversion with respect to the spatial contour at the each observation time point by using the following Legendre transformation formula:
$${a}_{n} ( t ) = \frac{2 n + 1}{2} {\int }_{0}^{π} f ( θ , t ) {P}_{n} ( cos θ ) sin θdθ$$
wherein aₙ(t) is a value of Legendre coefficient changed with time, n is an order value of different order vibrational modes, and f(θ, t) is a value of the relative displacement at different angles, wherein the different angles refer to a coordinate angle of the each observation point, Pₙ(cos θ) is Legendre polynomial, and the coordinate angle of the each observation point is θ.

17. The morphology determining system of corneal topography according to claim 13, wherein
the calculating module of the morphology determining system performs the mathematical function conversion with respect to the spatial contour at the each observation time point by using the following Fourier transformation formulas:
$${a}_{0} ( t ) = \frac{2}{π} {\int }_{0}^{π} f ( θ , t ) d θ$$
$${a}_{n} ( t ) = \frac{2}{π} {\int }_{0}^{π} f ( θ , t ) cos ( 2 n θ ) d θ$$
$${b}_{n} ( t ) = \frac{2}{π} {\int }_{0}^{π} f ( θ , t ) sin ( 2 n θ ) d θ$$
wherein a₀(t) is a value of Fourier coefficient applicable to a zeroth mode changed with time, aₙ(t) and bₙ(t) are respectively values of Fourier coefficients applicable to even order vibrational modes and odd order vibrational modes changed with time, and f(θ, t) is a value of the relative displacement at different angles, wherein the different angles refer to a coordinate angle of the each observation point, and the coordinate angle of the each observation point is θ.

18. The morphology determining system of corneal topography according to claim 14, wherein the one or more order vibrational modes calculated by the calculating module comprise at least first six order vibrational modes: a zeroth mode (M0), a first mode (M1), a second mode (M2), a third mode (M3), a fourth mode (M4), and a fifth mode (M5), wherein the zeroth mode (M0) highlights a feature of moving backward of an entire shape of the cornea, the first mode (M1) highlights a feature of rotating left and right of a shape of a ½ part of the cornea, the second mode (M2) highlights a feature of depression of a shape of a ⅓ part in a center of the cornea, the third mode (M3) highlights a feature of high-and-low distortion of a shape of a ¼ part adjacent to the center of the cornea, the fourth mode (M4) highlights a feature of depression of a shape of a ⅕ part in the center of the cornea, and the fifth mode (M5) highlights a feature of high-and-low distortion of a shape of a ⅙ part adjacent to the center of the cornea.

19. The morphology determining system of corneal topography according to claim 18, wherein the at least one reference cornea comprises a standard cornea, and a corresponding order vibrational mode of the standard cornea is calculated and established according to normal corneas of a plurality of normal persons substantially without eye diseases based on the parameter obtaining step (S1) and the conversion step (S2), and is stored in the calculating module or is accessible to the calculating module, and the calculating module implements the determining step (S3), comprising respectively determining, according to differences between the cornea and the standard cornea with respect to the zeroth mode (M0), the first mode (M1), the second mode (M2), the third mode (M3), the fourth mode (M4), and the fifth mode (M5), corneal features of the cornea with respect to the standard cornea based on the corneal features represented by the zeroth mode (M0), the first mode (M1), the second mode (M2), the third mode (M3), the fourth mode (M4), and the fifth mode (M5).

20. The morphology determining system of corneal topography according to claim 19, wherein based on a determining result of the corneal features, if the cornea of the target subject has a phenomenon of being relatively thin locally and protruding outwards or being depressed inwards with respect to the standard cornea, the calculating module determines in the determining step (S3) that the target subject suffers from keratoconus or corneal ectasia.

21. The morphology determining system of corneal topography according to claim 18, wherein the at least one reference cornea comprises an artificially set virtual cornea, and a corresponding order vibrational mode of the artificially set virtual cornea is preset, and is stored in the calculating module or is accessible to the calculating module, and the calculating module implements the determining step (S3), comprising respectively determining, according to differences between the cornea and the artificially set virtual cornea with respect to the zeroth mode (M0), the first mode (M1), the second mode (M2), the third mode (M3), the fourth mode (M4), and the fifth mode (M5), corneal features of the cornea with respect to the artificially set virtual cornea based on the corneal features represented by the zeroth mode (M0), the first mode (M1), the second mode (M2), the third mode (M3), the fourth mode (M4), and the fifth mode (M5).

22. The morphology determining system of corneal topography according to claim 14, wherein the at least one reference cornea comprises one or more specific morphological corneas, and respective corresponding order vibrational modes of the one or more specific morphological corneas are respectively calculated and established substantially according to each of the one or more specific morphological corneas based on the parameter obtaining step (S1) and the conversion step (S2), and
at least one corresponding order vibrational mode of different corresponding order vibrational modes of each of the one or more specific morphological corneas has at least one curve feature, and the corresponding order vibrational modes of the one or more specific morphological corneas or the curve features of the one or more specific morphological corneas are stored in the calculating module or are accessible to the calculating module, wherein
the calculating module implements the determining step (S3), comprising determining, through comparison, whether the one or more order vibrational modes of the cornea have the corresponding curve features in the respective corresponding vibrational modes, and
when the one or more order vibrational modes of the cornea have the curve features above a preset threshold, the calculating module determines in the determining step (S3) that the cornea is a corresponding morphological cornea of the one or more specific morphological corneas having the curve features.

23. The morphology determining system of corneal topography according to claim 14, wherein the at least one reference cornea comprises one or more specific morphological corneas, and respective corresponding order vibrational modes of the one or more specific morphological corneas are respectively calculated and established substantially according to each of the one or more specific morphological corneas based on the parameter obtaining step (S1) and the conversion step (S2), and are stored in the calculating module or are accessible to the calculating module, and wherein
the calculating module implements the determining step (S3), comprising comparing each vibrational mode of the one or more order vibrational modes of the cornea with a corresponding vibrational mode of each of the one or more specific morphological corneas, and when the one or more order vibrational modes of the cornea accord with curve forms above a preset mode quantity, the determining step (S3) determines that the cornea is a corresponding morphological cornea of the one or more specific morphological corneas according with the curve forms.
